(19) United States
(12) Patent Application Publication
(10) Pub. No.: US 2024/0148654 A1
(43) Pub. Date: May 9, 2024

(54) LYOPHILIZED AND STABILIZED LIVE ATTENUATED FORMULATED VACCINE AGAINST TULAREMIA

(71) Applicant: Southwest Research Institute, San Antonio, TX (US)

(72) Inventors: Kenneth H. CARSON, San Antonio, TX (US); Spring M. CABINESS, Mico, TX (US); Amy M. DE LOS SANTOS, San Antonio, TX (US); Karl KLOSE, San Antonio, TX (US)

(21) Appl. No.: 18/503,901

(22) Filed: Nov. 7, 2023

Related U.S. Application Data
(60) Provisional application No. 63/382,667, filed on Nov. 7, 2022.

(51) Int. Cl. A61K 9/19; A61K 39/02; A61P 37/04

(52) U.S. Cl. CPC A61K 9/19; A61K 39/0208; A61P 37/04

(57) ABSTRACT

A lyophilized stabilized formulation containing a live attenuated vaccine strain against tularemia.

### GOVERNMENT SUPPORT

This invention was made with U.S. Government support under Agreement No. W15QKN-16-0-1002 awarded by the ACC-NJ to the MCDC. The Government has certain rights in the invention.

### FIELD

The present invention is directed at a lyophilized stabilized formulation containing a live attenuated vaccine strain against tularemia.

### BACKGROUND

Tularemia, is caused by the gram-negative intracellular pathogen Francisella tularensis. This bacterium was first identified in 1912. Francisella tularensis is currently listed as a category A selected agent. Since the discovery of this pathogen, at least four (4) subspecies have been identified that exhibit distinct virulence and biochemical profiles. Human disease is primarily associated with two F. tularensis subspecies: the highly virulent F. tularensis subsp. tularensis (Ftt) which is found in North America and the moderately virulent F. tularensis subsp. holarctica which is endemic throughout the Northern Hermisphere. See, e.g., Francisella tularensis in the United States, Farlow et al, Emerging Infectious Diseases, Vol. 11, No. 12 (Dec. 2005).

Currently, the only vaccine used to treat tularemia caused by Francisella tularensis is the Live Vaccine Strain (LVS), an attenuated type B (F. tularensis subsp. Holarctica) strain that does not appear to cause disease in humans but has unknown genetic stability and safety which makes it a poor candidate for FDA approval. As such, F. tularensis is classified as a category A select agent and is a threat to the general public as well as the warfighter.

Other relatively more recent efforts have reported on the use of an engineered form of F. novicida (Fn) in the form of Fn-igID OAg^{FTT} as a candidate for a tularemia vaccine. See, e.g., Engineered OAg^{FTT} expression in F. novicida, X. Zogay, A. Adelani, J. Nguyen, P. Chu, K. Klose, 9^{th} International Conference on Tuleremia, October 2018.

A need remains to produce an FDA-approved formulated vaccine against F. tularensis, allowing intradermal delivery and providing relatively high protective immunity with storage stability.

### BRIEF DESCRIPTION OF THE DRAWINGS

The lyophilized stabilized formulation containing a live attenuated vaccine strain against tularemia is further described herein with reference to the accompanying drawings identified below.

FIG. 1 illustrates the survival of F344 rats after intradermal administration of the 10% TMC stabilized lyophilized vaccine or vaccines and boosts upon challenge with a F. tularensis Schu S4 mutant strain by the intratracheal route.

FIG. 2A plots the level of polymorphonuclear leukocytes populations in the lungs of 10% TMC lyophilized KKF768 and mock vaccinated rats at days 3, 5, and 7, following an intratracheal challenge with a potentially lethal dose of Ft SCHU S4.

FIG. 2B plots the level of natural killer cell populations in the lungs of 10% TMC lyophilized KKF768 and mock vaccinated rats at days 3, 5, and 7, following an intratracheal challenge with a potentially lethal dose of Ft SCHU S4.

FIG. 2C plots the level of CD43hi monocytes in the lungs of 10% TMC lyophilized KKF768 and mock vaccinated rats at days 3, 5, and 7, following an intratracheal challenge with a potentially lethal dose of Ft SCHU S4.

FIG. 2D plots the level of His48hi monocytes in the lungs of 10% TMC lyophilized KKF768 and mock vaccinated rats at days 3, 5, and 7, following an intratracheal challenge with a potentially lethal dose of Ft SCHU S4.

FIG. 3A plots the level of CD4+Th cells in the lungs of 10% TMC lyophilized KK768 and mock vaccinated rats at days 3, 5, and 7 following an intratracheal challenge with a potentially lethal does of Ft SCHU S4.

FIG. 3B plots the level of CD8+Tc cells in the lungs of 10% TMC lyophilized KK768 and mock vaccinated rats at days 3, 5, and 7 following an intratracheal challenge with a potentially lethal dose of Ft SCHU S4.

FIG. 3C plots the level of B cells in the lungs of 10% TMC lyophilized KK768 and mock vaccinated rats at days 3, 5, and 7 following an intratracheal challenge with a potentially lethal dose of Ft SCHU S4.

FIG. 4A plots the level of Th1 cells in the lungs of 10% TMC lyophilized KK768 and mock vaccinated rats at days 3, 5, and 7 following an intratracheal challenge with a potentially lethal dose of Ft SCHU S4.

FIG. 4B plots the level of Th2 cells in the lungs of 10% TMC lyophilized KK768 and mock vaccinated rats at days 3, 5, and 7 following an intratracheal challenge with a potentially lethal dose of Ft SCHU S4. F

FIG. 4C plots the level of T171 cells in the lungs of 10% TMC lyophilized KK768 and mock vaccinated rats at days 3, 5, and 7 following an intratracheal challenge with a potentially lethal dose of Ft SCHU S4.

FIG. 4D plots the level of Tc 1 cells in the lungs of 10% TMC lyophilized KK768 and mock vaccinated rats at days 3, 5, and 7 following an intratracheal challenge with a potentially lethal dose of Ft SCHU S4.

FIG. 4E plots the level of Tc2 cells in the lungs of 10% TMC lyophilized KK768 and mock vaccinated rats at days 3, 5, and 7 following an intratracheal challenge with a potentially lethal dose of Ft SCHU S4.

FIG. 4F plots the level of Tc17 cells in the lungs of 10% TMC lyophilized KK768 and mock vaccinated rats at days 3, 5, and 7 following an intratracheal challenge with a potentially lethal dose of Ft SCHU S4.

### DETAILED DESCRIPTION

The present invention starts with consideration of F. novicida (Fn), a closely related species to Francisella tularensis subsp. tularensis (Ftt), that is avirulent in humans. Namely, Fn with mutation igID, the T6SS gene required for phagosome escape and intramacrophage replication (Fn-igID) is configured to express the lipopolysaccharide (LPS) 0-Antigen from FTT (Ftt(OAg^{FTT}). This is preferably achieved by removal of the entire OAg cluster consisting of 12 genes from Fn-igID followed by the stepwise replacement of the OAg cluster consisting of 15 genes from Ft subsp. holarctica LVS streain, which express OAg^{FTT}. The resultant strain, KKF768 (Fn-igID OAg^{FTT}) expresses OAg^{FTT} attached to its LPS.

For lyophilization of KKF768, a preferred stabilized formulation was employed that contained the stabilizer mix of 10.0% Trehaolose (w/v), 5.0% Mannitol (w/v) and 0.2% Cysteine (w/v), as cryoprotecatants, hereinafter the mix is identified as “10% TMC.” An aqueous solution of this stabilized mix was prepared that preferably contained a suspension of 1×10¹⁰ cfu/mL of KKF768. Lyophilization then followed.

A preferred procedure of lyophilization is as follows. Fn-igID 0Ag m is suspended in the 10% TMC solution is aliquoted (e.g., 0.5 ml) into 5.0 ml (20 mm×40 mm) vials with a fluoropolymer stopper (e.g., FluroTec® stopper) and then transferred to a Labconco Freezone 12L with a stoppering tray dryer attachment. Table 1 below provides the preferred range of parameters for lyophlization used to provide the 10% TMC lyophilized formulation:

TABLE 1
Preferred Lyophilization Parameters

| | | | | | Pressure | | |
|---|---|---|---|---|---|---|---|
| | | Shelf | Soak | Ramping | Setpoint | | |
| | | Temperature | Time | Rate | (micron | | |
| Segment | | Setpoint (° C.) | (hours) | (° C./hour) | or PSIA) | | Mbar |
| Load | Product | 5.0 | 1.0 | | | | |
| | Loading | | | | | | |
| 1 | Freezing | −40.0 | 3.0 | 60.0¹ | | | |
| 2 | Primary | −30.0 | 48.0 | 30.0² | 40 | micron | 0.52 |
| | Drying | | | | | | |
| 3 | Secondary | 5.0 | | 30.0³ | | | |
| | Drying | | | | | | |
| 4 | Stoppering | 5.0 | | | 14.7 | PSIA | No |
| | | | | | | | vacuum |

¹The product is cooled to −40.0° C. from 5.0° C. at the preferred rate of 60.0° C. per hour or in the range of 55.0° C. per hour to 65.0° C. per hour.
²The frozen product at −40.0° C. is raised to −30.0° C. at the preferred rate of 30.0° C. per hour or in the range of 25.0° C. per hour to 35.0° C. per hour.
³The product at −30.0° C. is raised to 5.0° C. at the preferred rate of 30.0° C. per hour or in the range of 25.0° C. per hour to 35.0° C. per hour.

From the above, it can be appreciated that the freezing preferably occurs at a temperature range of −45.0° C. to −35.0° C. for a period of time in the range of 2.5 hours to 3.5 hours, primary drying occurs at a temperature of −35.0° C. to −25.0° C. at 0.45 Mbar to 0.55 Mbar for a time in the range of 40-55 hours, and secondary drying occurs at a temperature of 0° C. to 10° C., the chamber is then purged with nitrogen, and the sample can be stoppered in the vials and removed.

Viability of the lyophilized KK768 is initially summarized below in Table 2:

TABLE 2
Viability of KKF768 Lyophilized in 10% TMC and
Stored at Various Temperatures and Times^{1,2}

| Storage | | Week | Week | Week | Week | Week | Week |
|---|---|---|---|---|---|---|---|
| Condition | | 1 | 2 | 4 | 8 | 12 | 24 |
| −20° | C. | — | — | 9.62 | 9.35 | 9.33 | 9.42 |
| 4° | C. | 9.59 | 9.47 | 9.10 | 8.58 | 8.1 | — |

| | | | | | | |
|---|---|---|---|---|---|---|
| 25° C./60% | 8.95 | 7.29 | 6.96 | — | — | — |
| RH | | | | | | |

¹Pre-lyophilized 10.13; Post-lyophilized 9.74 (−0.39).
²Viability is reported in log₁₀ of colony forming units per vial. 0.5 mL of suspension lyophilized in each vial.

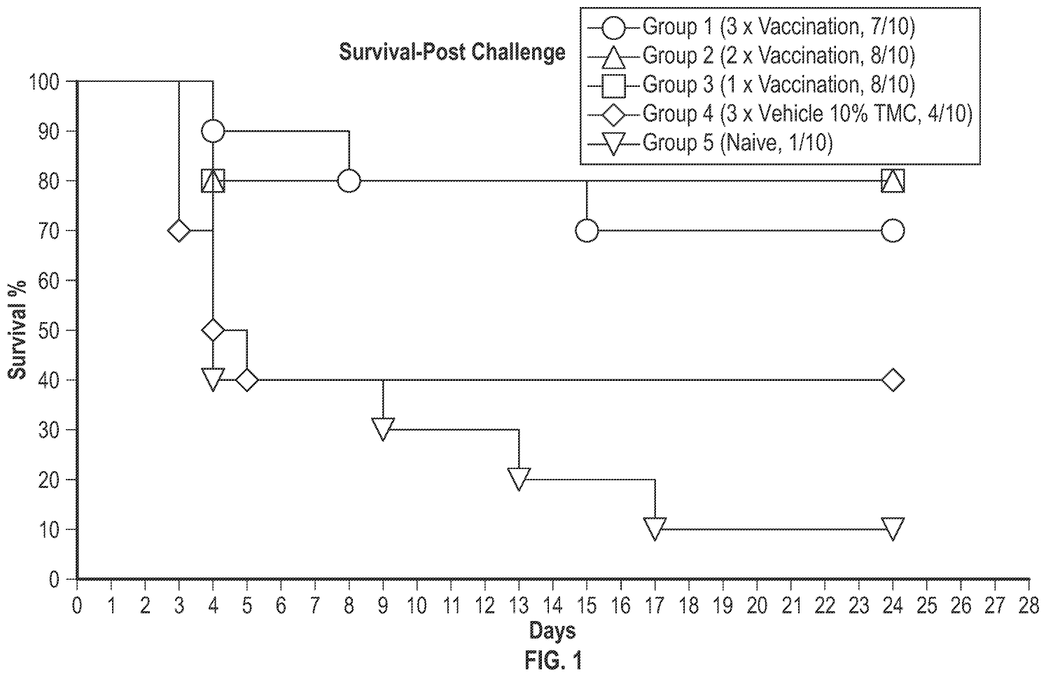

FIG. 1 illustrates the survival of F344 rats after intradermal administration of the 10% TMC stabilized lyophilized vaccine or vaccines and boosts upon challenge with a F. tularensis Schu S4 mutant strain by the intratracheal route.

Correlates of immunity were next evaluated for the 10% TMC lyophilized KKF768 and as illustrated in the data below immune cells upregulated upon vaccination resulting in protective immunity after vaccination and boost.

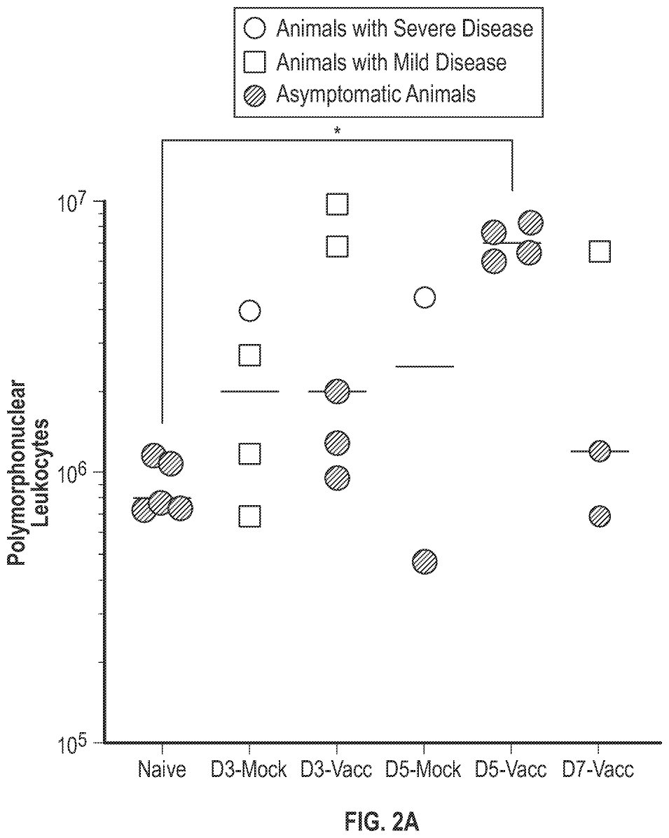

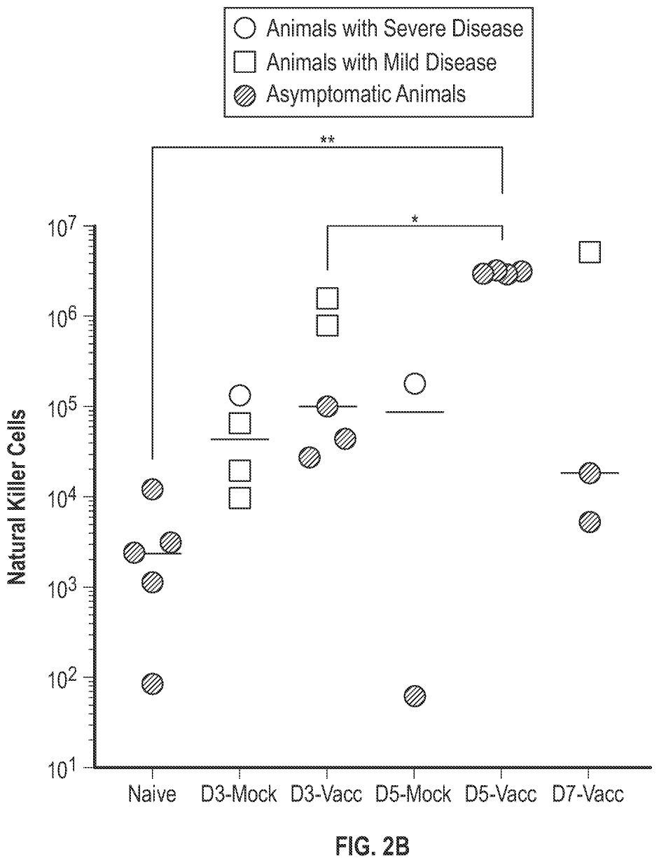

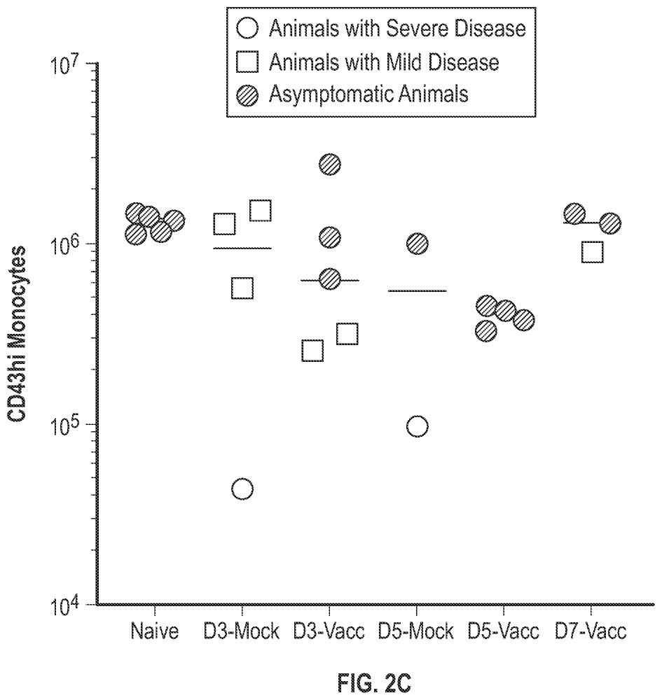

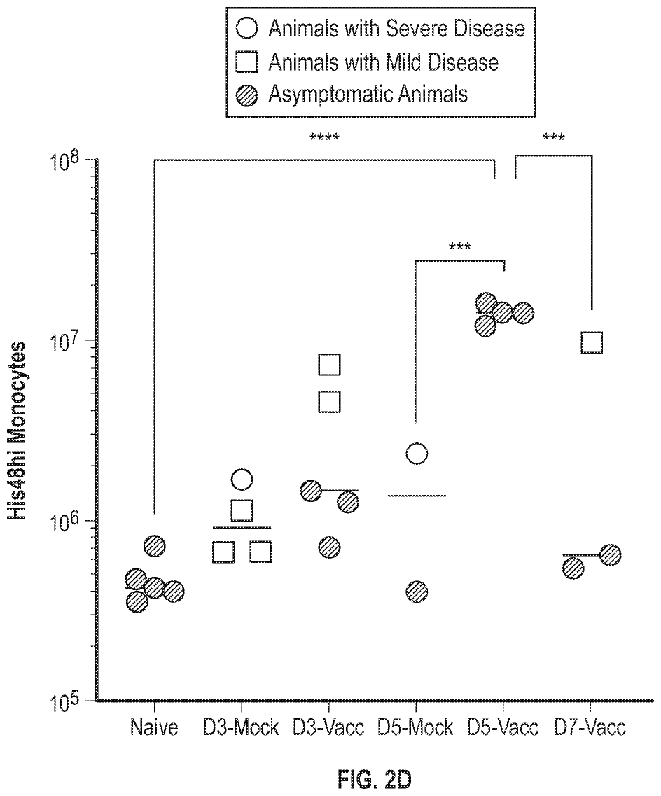

FIG. 2A plots the level of polymorphonuclear leukocytes populations in the lungs of 10% TMC lyophilized KKF768 and mock vaccinated rats at days 3, 5, and 7, following an intratracheal challenge with a potentially lethal dose of Ft SCHU S4. FIG. 2B plots the level of natural killer cell populations in the lungs of 10% TMC lyophilized KKF768 and mock vaccinated rats at days 3, 5, and 7, following an intratracheal challenge with a potentially lethal dose of Ft SCHU S4. FIG. 2C plots the level of CD43hi monocytes in the lungs of 10% TMC lyophilized KKF768 and mock vaccinated rats at days 3, 5, and 7, following an intratracheal challenge with a potentially lethal dose of Ft SCHU S4. FIG. 2D plots the level of His48hi monocytes in the lungs of 10% TMC lyophilized KKF768 and mock vaccinated rats at days 3, 5, and 7, following an intratracheal challenge with a potentially lethal dose of Ft SCHU S4.

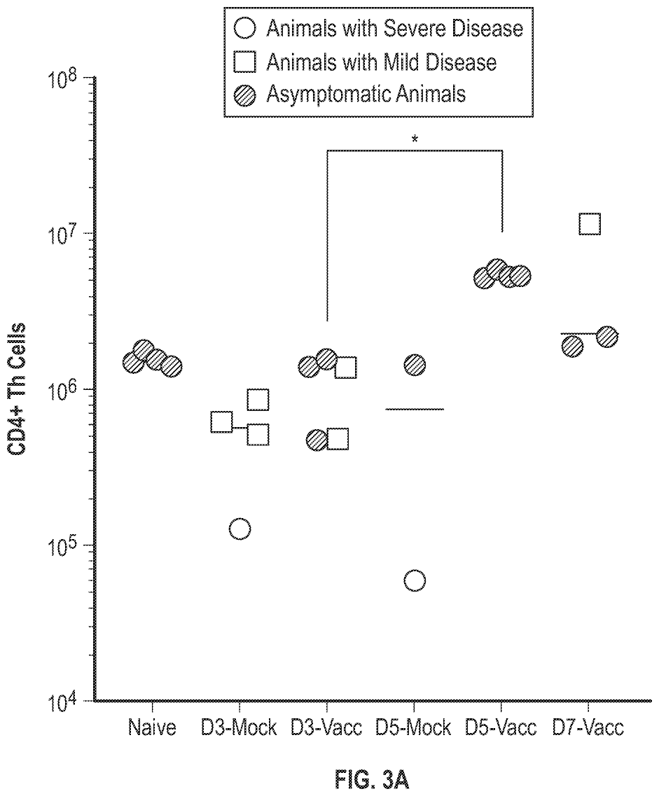

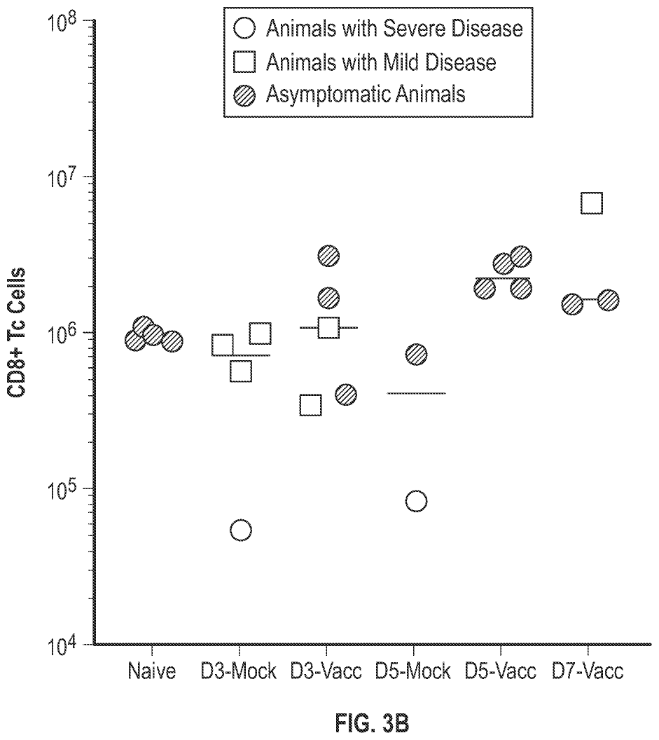

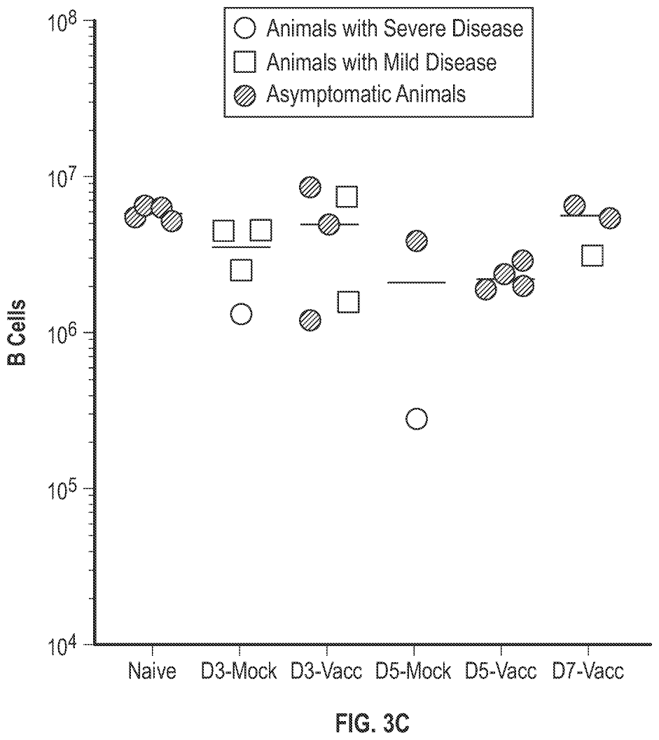

FIG. 3A plots the level of CD4+Th cells in the lungs of 10% TMC lyophilized KK768 and mock vaccinated rats at days 3, 5, and 7 following an intratracheal challenge with a potentially lethal does of Ft SCHU S4. FIG. 3B plots the level of CD8+Tc cells in the lungs of 10% TMC lyophilized KK768 and mock vaccinated rats at days 3, 5, and 7 following an intratracheal challenge with a potentially lethal dose of Ft SCHU S4. FIG. 3C plots the level of B cells in the lungs of 10% TMC lyophilized KK768 and mock vaccinated rats at days 3, 5, and 7 following an intratracheal challenge with a potentially lethal dose of Ft SCHU S4.

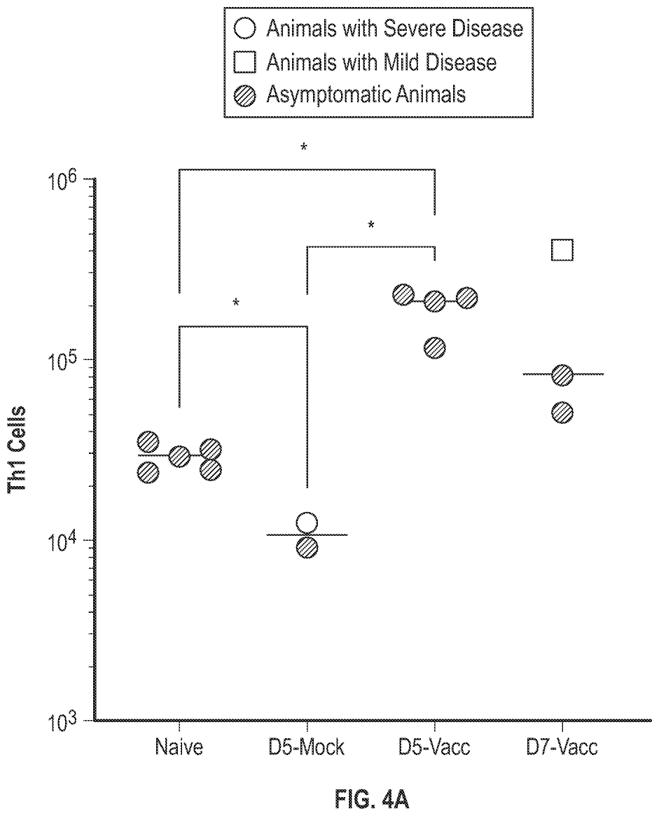

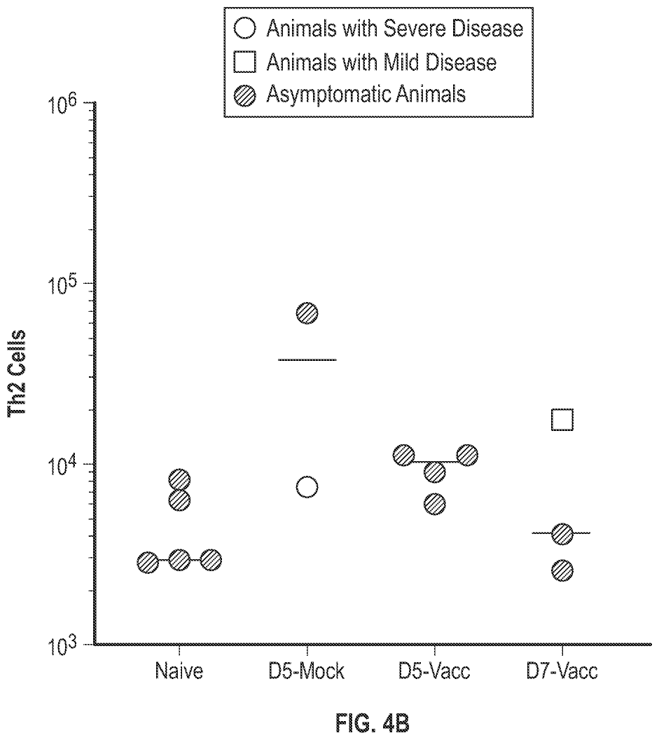

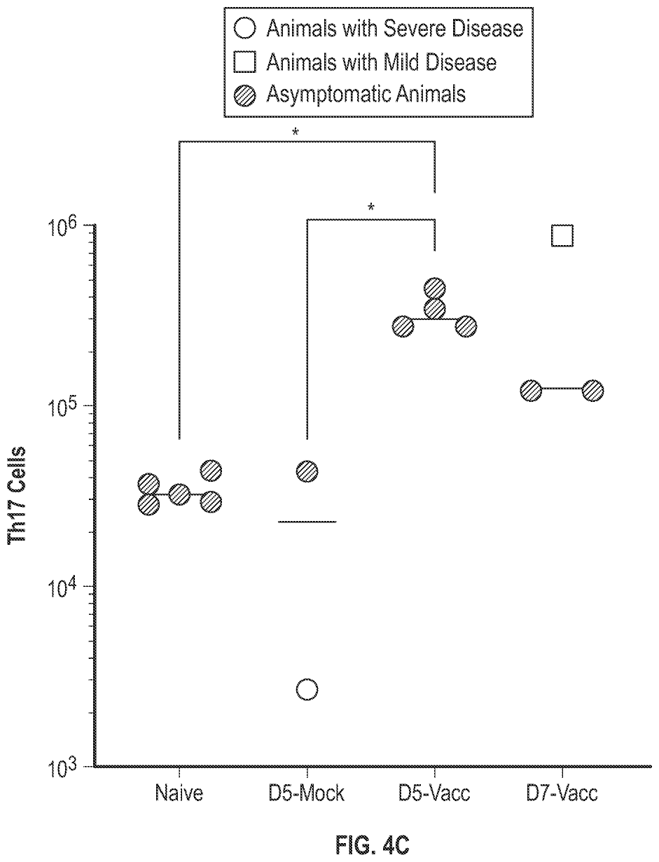

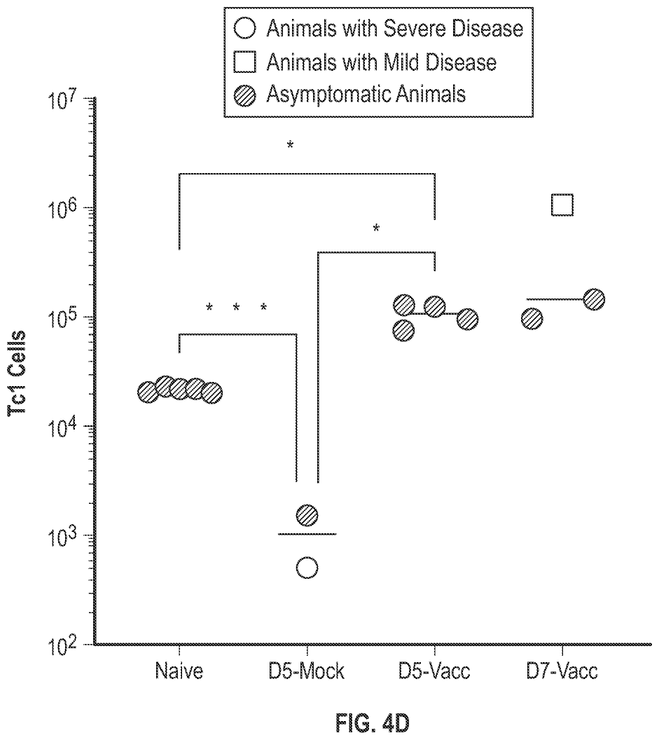

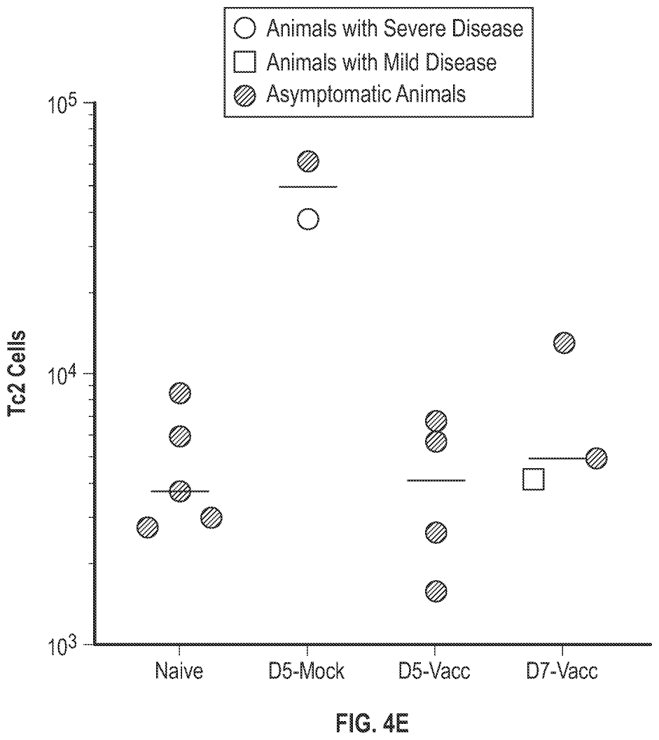

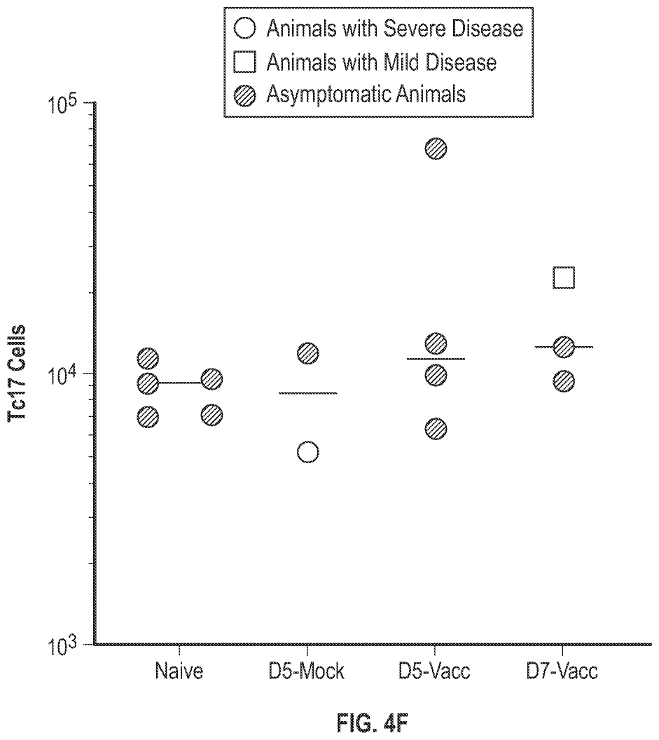

FIG. 4A plots the level of Th1 cells in the lungs of 10% TMC lyophilized KK768 and mock vaccinated rats at days 3, 5, and 7 following an intratracheal challenge with a potentially lethal dose of Ft SCHU S4. FIG. 4B plots the level of Th2 cells in the lungs of 10% TMC lyophilized KK768 and mock vaccinated rats at days 3, 5, and 7 following an intratracheal challenge with a potentially lethal dose of Ft SCHU S4. FIG. 4C plots the level of T171 cells in the lungs of 10% TMC lyophilized KK768 and mock vaccinated rats at days 3, 5, and 7 following an intratracheal challenge with a potentially lethal dose of Ft SCHU S4. FIG. 4D plots the level of Tc1 cells in the lungs of 10% TMC lyophilized KK768 and mock vaccinated rats at days 3, 5, and 7 following an intratracheal challenge with a potentially lethal dose of Ft SCHU S4. FIG. 4E plots the level of Tc2 cells in the lungs of 10% TMC lyophilized KK768 and mock vaccinated rats at days 3, 5, and 7 following an intratracheal challenge with a potentially lethal dose of Ft SCHU S4. FIG. 4F plots the level of Tc17 cells in the lungs of 10% TMC lyophilized KK768 and mock vaccinated rats at days 3, 5, and 7 following an intratracheal challenge with a potentially lethal dose of Ft SCHU S4.

As may now be appreciated from the above, the present disclosure stands directed to an immunogenic agent for use in the prevention or treatment of infection by Francisella tularensis in an animal, the immunogenic agent comprising a lyophilized and stabilized formulation of Fn-igID OAg^{FTT}. The animal may be a human. A pharmaceutical composition may also be provided containing the lyophilized stabilized formulation of Fn-igID OAg^{FTT}. In process form the present disclosure provides a method of producing an immunogenic agent for use in the prevention or treatment of infection by Francisella tularensis in an animal comprising the steps of supplying the immunogenic agent comprising Fn-igID OAg^{FTT} and subjecting said immunogenic agent to lyophilization and recovering a lyophilized and stabilized form of said immunogenic agent. The present disclosure also relates to a method of treatment of an animal in need of treatment for prevention or infection by Francisella tularensis comprising administration of the immunogenic agent comprising the lyophilized stabilized formulation of Fn-igID OAg^{FTT}.

What is claimed is:

1. An immunogenic agent for use in the prevention or treatment of infection by Francisella tularensis in an animal, the immunogenic agent comprising a lyophilized and stabilized formulation of Fn-igID OAg^{FTT}.

2. The immunogenic agent of claim 1, wherein said stabilized formulation includes 10.0% Trehaolose (w/v), 5.0% Mannitol (w/v) and 0.2% Cysteine (w/v), as cryoprotectants.

3. The immunogenic agent of claim 1, wherein the animal is a human.

4. A pharmaceutical composition including an immunogenic agent for use in the prevention or treatment of infection by Francisella tularensis in an animal, the immunogenic agent comprising lyophilized stabilized formulation of Fn-igID OAg^{FTT}.

5. A method of producing an immunogenic agent for use in the prevention or treatment of infection by Francisella tularensis in an animal comprising the steps of:
supplying the immunogenic agent comprising Fn-igID OAg^{FTT};
subjecting said immunogenic agent to lyophilization and recovering a lyophilized and stabilized form of said immunogenic agent.

6. The method of claim 5, wherein said lyophilization comprises freezing said immunogenic agent at a temperature of −45.0° C. to −35.0° C. for a period of time in the range of 2.5 hours to 3.5 hours followed by drying at a temperature of −35.0° C. to −25.0° C. at 0.45 Mbar to 0.55 Mbar for a time in the range of 40-55 hours.

7. A method of treatment of an animal in need of treatment for prevention or infection by Francisella tularensis comprising administration of the immunogenic agent comprising the lyophilized stabilized formulation of Fn-igID OAg^{FTT}.
